(19) United States
(12) Patent Application Publication
(10) Pub. No.: US 2007/0066827 A1
(43) Pub. Date: Mar. 22, 2007

(54) Process for the preparation of aryl fused polycyclic lactams

(75) Inventors: Robert Eugene Handfield Jr, East Lyme, CT (US); Timothy J.N. Watson, Waterford, CT (US)

Correspondence Address: PFIZER INC, 150 EAST 42ND STREET, 5TH FLOOR - STOP 49, NEW YORK, NY, 10017-5612, US

(73) Assignee: PFIZER INC.

(21) Appl. No.: 10/547,619

(22) PCT Filed: Jan. 8, 2004
(86) PCT No.: PCT/IB04/00152

§ 371 Date: Dec. 5, 2006

Related U.S. Application Data
(60) Provisional application No. 60/440,266, filed on Jan. 15, 2003.

(51) Int. Cl. A61K 31/473; C07D 221/22

(52) U.S. Cl. 546/97

(57) ABSTRACT

A process for the preparation of aryl fused polycyclic lactams which are useful intermediates in the synthesis of aryl fused azapolycyclic compounds as agents for the treatment of neurological and psychological disorders.

### BACKGROUND OF THE INVENTION

The present invention relates to a process for the preparation of aryl fused polycyclic lactams of the formula 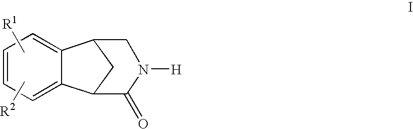
wherein R¹ and R² are as defined below.

Compounds of formula I are useful intermediates in the preparation of certain aryl fused azapolycyclic compounds which exhibit activity as agents for the treatment of neurological and psychological disorders.

U.S. patent application Ser. No. 09/514002, filed Feb. 25, 2000, discloses the preparation of 3-aminomethyl-indan-1-carboxylic acid methyl ester and the use of that compound as an intermediate in the synthesis of certain aryl fused azapolycyclic compounds.

U.S. patent application Ser. No. 10/124,135, filed Apr. 4, 2002 discloses the preparation of aryl-fused azapolycyclic compounds from intermediates having the formula I.

The synthesis, composition, and methods of use of certain aryl fused azapotycyclic compounds which exhibit activity as agents for the treatment of neurological and psychological disorders is disclosed in U.S. Pat. No. 6,410,550. The foregoing patent applications and patent are incorporated by reference herein in their entirety.

### SUMMARY OF THE INVENTION

The present invention relates to a process for preparing compounds having the formula 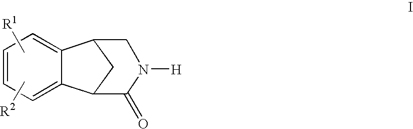
by hydrogenating a compound having the formula 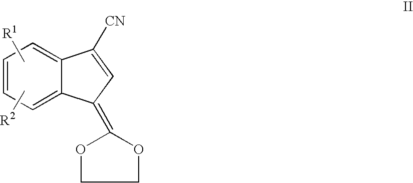
with hydrogen gas and an alcohol having the formula R³OH in the presence of a hydrogenation catalyst and an acid.

R¹ and R² are selected independently from hydrogen, C₁-C₅ alkyl, C₁-C₆ alkoxy, trifluoromethyl, halogen, sulfonyl alkyl, alkyamino, amide, ester, aryl-alkyl, hetero-alkyl and aryl-alkoxy;

or R¹ and R² together with the carbon atoms to which they are attached form a monocyclic or bicyclic ring;

and R³ is C₁ to C₆ alkyl.

The catalyst is about 5 % to about 10% palladium on carbon. Preferably the catalyst is about 5% palladium on carbon. In a preferred embodiment, R³ is C₁ or C₂ alkyl.

In the hydrogenation of compounds or formula II the nitrile group is reduced to the corresponding amino group.

The present invention provides a weight ratio of catalyst to compound of formula II of about 1:99 to about 10:90. Preferably the ratio is about 10:90.

Palladium on carbon catalysts are safely stored as a mixture of water and catalyst. Generally the mixture is comprised of about 30% to about 60% by weight of water, In a preferred embodiment of the present invention, the catalyst is comprised of about 50% by weight of water.

The acid is present at an equivalence ratio of acid to the amino group of about 1:1. Suitable acids include sulfuric acid, hydrochloric acid, phosphoric acid, trifluoroacetic acid, methane sulfonic acid, para-toluenesulfonic acid, acetic acid, formic acid, benzoic acid and salicylic acid. Preferably the add is sulfuric acid.

Intermediate compounds of the formula 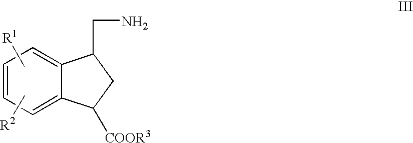
are cyclized to compounds of formula I by treatment with a base in a solvent comprising an alcohol of formula R³OH. Preferably the base is a Group I metal alkoxide. Most preferably the base is sodium tert-butoxide.

The cyclization of compounds of formula III into compounds of formula I is carried out in a solvent comprising an alcohol of formula R³OH wherein R³ is C₁ to C₆ alkyl. Preferably R³ is C₁ or C₂ alkyl.

In a preferred embodiment of the present invention, intermediate compounds of formula III are cyclized into compounds of formula I without prior isolation of intermediate III.

In another embodiment, intermediate compounds of formula III are isolated prior to conversion into compounds of formula I. Compounds of formula III may be isolated when R³ is C₃ to C₆ alkyl and the amino group is bound as an acid addition salt. Examples include, but are not limited to, the salts of p-toluene sulfonic acid, mandelic acid, salicylic acid, and tartaric acid.

In a preferred embodiment, the compound of formula I is selected from the group consisting of
10-aza-tricyclo[6,3,1,0,2,7] dodeca-2,4,6-triene-9one;
3-trifluoromethyl-10-aza-tricyclo[6,3,1,0^{2,7}]dodeca-2(7),3,5-triene9one;
(+)-3-trifluoromethyl-10-aza-tricyclo[6,3,1,0^{2,7}]dodeca-2(7),3,5-triene-9-one;
(−)-3-trifluoromethyl-10-aza-tricyclo[6,3,1,0^{2,7}]dodeca-2(7),3,5-triene-9-one;
3-fluoro-10-aza-tricyclo[6,3,1,0^{2,7}]dodeca-2(7),3,5-triene-9one;
(+)-3-fluoro-10-aza-tricyclo[6,3,1,0^{2,7}]dodeca-2(7),3,5-triene-9one; and
(−)-3-fluoro-10-aza-tricyclo[6,3,1,0^{2,7}]dodeca-2(7),3,5-triene.

### DETAILED DESCRIPTION OF THE INVENTION

The present invention provides a process for preparing compounds of the formula I by a sequence of reactions illustrated in Scheme I.

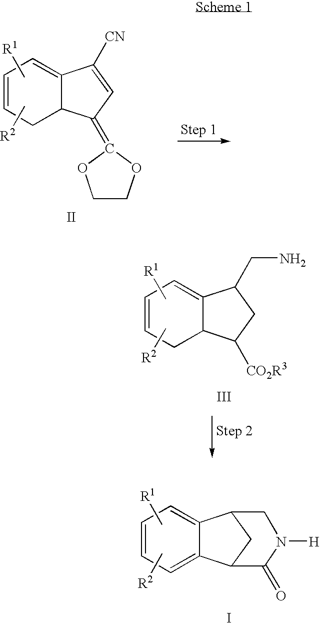

In Step 1, compounds of formula II are hydrogenated to the intermediate compound III in the presence of a hydrogenation catalyst, an alcohol of formula R³OH and an acid. The reaction involves reduction of the nitrile group to the corresponding amine, saturation of the indene ring, and conversion of the ketene acetal into the corresponding ester group of formula —CO₂R³.

R¹ and R² are selected independently from hydrogen, C₁-C₆ alkyl, C₁-C₅ alkoxy, trifluoromethyl, halogen, sulfonyl alkyl, alkyamino, amide, ester, aryl-alkyl, hetero-alkyl and aryl-alkoxy;

or R¹ and R² together with the carbon atoms to which they are attached form a monocyclic or bicyclic ring;

and R³ is C₁ to C₅ alkyl.

Hydrogenation catalysts suitable for the Step 1 conversion are generally stored for safety purposes as a mixture of catalyst and water. Generally, the hydrogenation catalyst is comprised of about 30% to about 60% by weight water for safe storage and handling.

Due to the inherent instability of compounds of formula II and III in the presence of water, it is an objective of the present invention to select a catalyst and hydrogenation conditions which impose limitations on the introduction of water. The catalyst is about 5% to about 10% palladium on carbon, preferably about 5% palladium on carbon with a weight ratio of catalyst to compound of formula II of about 1:99 to about 10:90. Preferably the ratio is about 10:90.

Generally, hydrogenation reactions of the type illustrated by Step 1 of Scheme 1 are conducted in the presence of an excess of acid. As used herein, the term excess acid refers to acid which is not bound as a salt with the amino group of formula III.

When the hydrogenation of compounds of formula II is carried out with an equivalence ratio of acid to amino group of about 2:1, the product yield is very low. Based upon the foregoing results, it is believed that compounds of formula II and formula III are unstable in the presence of excess acid.

In the present invention, the equivalence ratio of acid to amino Compound is 1:1 so that all of the acid is bound as a salt with the amino group of formula III.

The hydrogenation takes place in the presence of an acid such as sulfuric acid, acetic acid, formic acid, benzoic acid, or salicylic acid, preferably sulfuric acid, formic acid, acetic acid, or para-tuluenesulfonic acid, and most preferably sulfuric acid. Suitable solvents are methanol, ethanol, Isopropanol, butanol, propanol ethyl acetate, isopropyl acetate, tetrahydrofuran, toluene, or any mixture of these solvents, preferably methanol or ethanol. The reaction is carried out under a hydrogen atmosphere up to 7 atmospheres (approximately 100 psi), preferably 3 to 4 atmospheres (approximately 50 psi), for a time period of 1 to 48 hours preferably 12 hours. This affords a compound of formula II which may be a mixture of diastereomers.

The aforementioned conditions, which impose limitations on the introduction of water or acid into the reaction as illustrated by Step 1 of Scheme 1, provide a chemically stable environment resulting in improved yields of intermediate III.

As used herein, the term unstable refers to the potential for undesirable chemical side reactions which compounds of formula II or III undergo in the presence of water or excess acids. When a compound of formula II and or III undergoes undesirable side reaction, the yield of compound I is significantly reduced. The term chemically stable environment refers to the relatively low potential for compounds of formula II or III to undergo undesirable side reactions with water or acid.

Step 2 of Scheme 1 Is the formation of a lactam of formula I. The amino acid ester of formula (III) is treated with a base such as sodium tert-butoxide, sodium methoxide, sodium ethoxide, potassium tert-butoxide, potassium methoxide, and potassium ethoxide, sodium carbonate, potassium carbonate, cesium carbonate, sodium hydride, triethylamine, methylimidazole, lutdine, pyridine, methylmorpholine, ethylmorpholine or dissdopropylethylamine. Preferably the base is a Group I metal alkoxide. Most preferably the base is sodium tert-butoxide. The alkoxide base preferably has a very low sodium hydroxide content.

Suitable solvents are methanol, ethanol, isopropanol, ethyl acetate, acetonitrile, toluene, or a mixture of any of the previously mentioned solvents, preferably methanol or a mixture of methanol and ethyl acetate. The reaction is conducted at a temperature of 0° to 120° C., preferably at room temperature. The reaction is extended for a time period of 0.5 hour to 72 hours, preferably 6 hours, to afford a compound of formula (I).

Based upon the aforementioned side reactions of compounds of formula II and III, the solvent for Step 2 contains a minimal quantity of water.

In a preferred embodiment, intermediate compounds of formula III are not isolated prior to the cyclization of Step 2. The base is added directly to a filtered solution of the intermediate III with subsequent cyclization to the lactam I.

In another embodiment the intermediate compound III may be isolated wherein R³ is C₃ to Cₐ and the amino group is bound as an acid addition salt. Examples include but are not limited to the salts of p-toluene sulfonic acid, mandelic acid salicylic acid, and tartaric acid.

The intermediate III, either in the form of an isolated compound or as a solution without prior isolation, may be converted into the lactam I according to the aforementioned cyclization conditions.

Compounds of formula I are useful intermediates in the synthesis of aryl fused azapolycyclic compounds exhibiting activity in the treatment of neurological and psychological disorders.

The conversion of compounds of formula II into alyl fused azapolycyclic compounds of formula IV is illustrated in Scheme 2 . 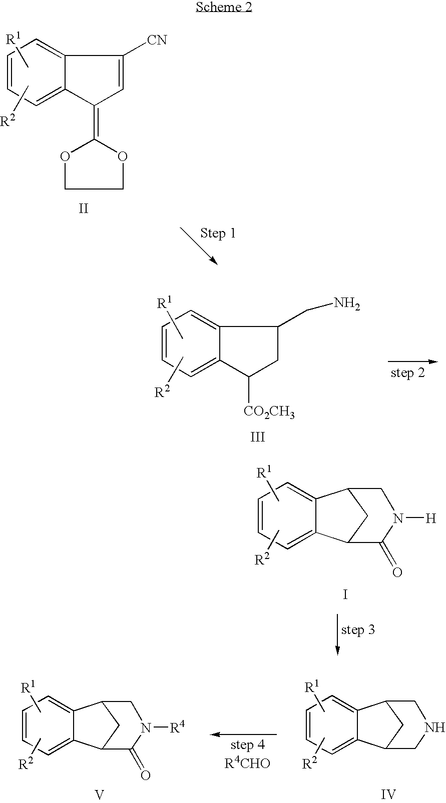
wherein R⁴ is hydrogen, C₁-C₆ alkyl, unconjugated C₃-C₆ alkenyl, benzyl or alkoxy C₁ -C₆.

In step 1, hydrogenation of compounds of formula 11 yields the intermediate II which is cyclized in step 2 with sodium t-butoxide in methanol to form the lactam formula I. The carbonyl function is reduced in step 3 with sodium borohydride-borontrifluoride giving the aryl fused azapolycylic compound of formula IV.

Examples of specific compounds of the formula IV are the following compounds:
4ethynyl-5chloro-10-aza-tricyclo[6,3,0^{2,7}]dodeca-2(7),3,5-triene;
3trifluoromethyl-10aza-tricyclo[6,3,0^{2,7}]dodeca-2(7),3,5-triene;
4,5-bistrifluoromethyl-10aza-tricyclo[6,3,1,0^{2,7}]dodeca-2(7),3,5triene;
4-choro-5-trifluoromethyl-10aza-tricyclo[6,3,1,0 ^{2,7}]dodeca-2(7),3,5-triene;
4-amino-10-aza-tricyclo[6,3,1,0^{2,7}]dodeca-2(7),3,5-triene;
4-nitro-10-aza-tricyclo[6,3,0^{2,7}]dodeca-2(7),3,5-triene;
4-methyl-10-aza-tricyclo[6,3,0^{2,7}]dodeca-2(7),3,5-triene;
4-fluoro-10-aza-tricyclo[6,3,0^{2,7}]dodeca-2(7),3,5-triene;
4-trifluoromethyl-10-aza-tricyclo[6,3,0^{2,7}]dodeca2(7),3,5-triene; and
4,5-difluoro-10-aza-tricyclo[6,3,0^{2,7}]dodeca2(7),3,5-triene;
4-nitro-10-aza-tricyclo[6,3,1,0^{2,7}]dodeca2(7),3,5-triene;
4,5-dinitro-10-aza-tricyclo[6,3,1,0^{2,7}]dodeca2(7),3,5-triene;
4,5-dichloro-10-aza-tricyclo[6,3,1,0^{2,7}]dodeca2(7),3,5-triene;
(+)-3-trifluoromethyl-10-aza-tricyclo[6,3,1,0^{2,7}]dodeca2(7),3,5-triene;
(−)-3-trifluoromethyl-10-aza-tricyclo[6,3,1,0^{2,7}]dodeca2(7),3,5-triene;
3-fluoro-10-aza-tricyclo[6,3,1,0^{2,7}]dodeca2(7),3,5-triene;
(+) 3-fluoro-10-aza-tricyclo[6,3,1,0^{2,7}]dodeca2(7),3,5-triene;
(−) 3-fluoro-10-aza-tricyclo[6,3,1,0^{2,7}]dodeca2(7),3,5-triene;
4-ethynyl-5-fluoro-10-aza-tricyclo[6,3,1,0^{2,7}]dodeca2(7),3,5-triene;
(+)-4-ethynyl-5-fluoro-10-aza-tricyclo[6,3,1,0^{2,7}]dodeca2(7),3,5-triene;
(−)-4-elhynyl-5-fluoro-10-aza-tricyclo[6,3,1,0^{2,7}]dodeca2(7),3,5-triene;
4-fluoro-5-trifluoromethyl-10-aza-tricyclo[6,3,1,0^{2,7}]dodeca2(7),3,5-triene;
(+)-4-fluoro-5-trifluoromethyl-10-aza-tricyclo[6,3,1,0^{2,7}]dodeca2(7),3,5-triene; and
(−)-4-fluoro-5-trifluoromethyl-10-aza-tricyclo[6,3,1,0^{2,7}]dodeca2(7),3,5-triene; and pharmaceutically acceptable salts thereof.

In step 4 of Scheme 2, derivatives of formula IV are prepared by condensing the secondary amino of formula IV with an aldehyde of formula R⁴CHO.

Compounds of formula V bind to neuronal nicotinic acetylcholine specific receptor sites and are useful in modulating cholinergic function. Such compounds are useful in the treatment of inflammatory bowel disease (including but not limited to ulcerative colitis, pyoderma gangrenosum and Crohn's disease), irritable bowel syndrome, spastic dystonia, chronic pain, acute pain, celiac sprue, pouchitis, vasoconstriction, anxiety, panic disorder, depression, bipolar disorder, autism, sleep disorders, jet lag, amyotrophic lateral sclerosis (ALS), cognitive dysfunction, hypertension, bulimia, anorexia, obesity, cardiac arrythmias, gastric acid hypersecretion, ulcers, pheochromocytoma, progressive supranuclear palsy, chemical dependencies and addictions (e.g. , dependencies on, or addictions to nicotine (and/or tobacco products), alcohol, benzodiazepines, barbiturates, opioids or cocaine), headache, migraine, stroke, traumatic brain injury (TBI), obsessive-compulsive disorder (OCD), psychosis, Huntington's chorea, tardive dyskinesia, hyperkensia, dyslexia, schizophrenia, multi-infarct dementia, age-related cognitive decline, epilepsy, including petit mal absence epilepsy, senile dementia of the Alzheimer's type (AD), Parkinson's disease (PD), attention deficit hyperactivity disorder (ADHD) and Torette's Syndrome.

The compounds of formula V, and their pharmaceutically acceptable salts (hereafter “the active compounds”) can be administered via either the oral, transdermal (e.g., through the use of a patch), intranasal, sublingual, rectal, parenteral or topical routes. Transdermal and oral administration are preferred. These compounds are, most desirably, administered in dosages ranging from about 0.01 mg up to about 1500 mg per day, preferably from about 0.1 to about 300 mg per day in single or divided doses, although variations will necessarily occur depending upon the weight and condition of the subject being treated and the particular route of administration chosen. However, a dosage level that is in the range of about 0.001 mg to about 10 per kg of body weight per day is most desirably employed. Variations may nevertheless occur depending upon the weight and condition of the persons being treated and their individual responses to said medicament, as well as on the type of pharmaceutical formulation chosen and the time period and interval during which such administration is carried out in some instances, dosage levels below the lower limit of the aforesaid range may be more than adequate, while in other cases still larger dose may be employed without causing any harmful side effects, provided that such larger doses are first divided into several small doses for administration throughout the day.

The active compounds can be administered alone or in combination with pharmaceutically acceptable carriers or diluents by any of the several routes previously indicated. More particularly, the active compounds can be administered in a wide variety of different dosage forms, e.g., they may be combined with various pharmaceutically acceptable inert carriers in the form of tablets, capsules, transdermal patches, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents. In addition, oral pharmaceutical compositions can be suitably sweetened and/or flavored. In general, the active compounds are present in such dosage forms at concentration levels ranging from about 5.0% to about 70% by weight.

For oral administration, tablets may contain a variety of excipients, disintegrants, lubricating agents, and fillers.

Aqueous suspensions for oral administration may be embodied with flavor, coloring matter, and diluent.

For parenteral administration, a solution of the active compound may be suitably buffered and may be diluted with a vegetable oil or propylene glycol.

The following examples are provided for the purpose of further illustration and are not intended to limit the scope of the invention.

### EXAMPLE 1

### 3-Aminomethyl-indan-1-carboxylic acid methyl ester

A first reactor was charged with 3-[1,3]dioxolan-2-ylidene-3H-indene-1-carbonitrile (47.3 kg 223.9 moles) and 5% palladium on carbon (50% water: 4.7 kg). Methanol (126 kg) was charged to reactor 2 and cooled to 0° C. to 5° C. Added sulfuric acid (22.3 kg) to the methanol in reactor 2 at 0° C. to 5° . Held this acid solution at 0° C. to 5° C. until needed. Charged methanol (136.5 kg) to reactor 1 at 0° to 5° C. Both reactors were purged independently to minimize the exposure time of the ketene acetal to the acid and water from the catalyst. Now charged the methlanol/sulfuric acid solution in reactor 2 to the contents in reactor 1 at 0° C. to 56° C. and the hydrogen was introduced immediately to begin the hydrogenation. The contents in reactor 1 were than hydrogenated at 50 psig starting at a temperature of 0° C. and slowly ramping the temperature up to 50° C. to 55° C. until the uptake of hydrogen ceased. The reaction was then sampled for reaction completion and once deemed complete, reactor 1 was purged with nitrogen and cooled to 200° C. to 25° C. The contents in reactor 1 were then filtered to remove the spent catalyst and the catalyst cake rinsed with methanol (165 kg). The filtrate from reactor 1 and the methanol rinse were then held without isolation for use in the next step.

Example 2

### 10-Aza-tricyclo[6,3,1,0,2,7]dodeca-2,4,6-triene-9-one

The methanolic solution obtained in Example 1 (539 L, 46 kg Theory) was concentrated to a volume of 114 L in reactor 1. Methanol (460L) and 25% Sodium methoxide/methanol solution (124 L) were charged to reactor 2 at 15° C. to 25° C. The contents of reactor 1 were slowly charged into reactor 2 at 15° C. to 250° C. Rinsed reactor 1 with methanol (19 L) and transfer the rinse to reactor 2 at 15° C. to 25° C. The contents in reactor 2 were stirred for 15 hours at 15° C. to 25° C. The reaction was sampled and once deemed complete, 85% phosphoric acid (20 L) was added in small portions to achieve a pH of 4.5 to 5 at 15° C. to 25° C. The contents in reactor 2 were concentrated to 148 L and water (322 L) was then added to reactor 2 at 15° C. to 25° C. The contents in reactor 2 were concentrated to 367 L and methylene chloride then charged to reactor 2 at 15° C. to 25° C. The contents in reactor 2 were then stirred 30 minutes at 15° C. to 25° C. and then allowed to settle for 45 minutes. The layers were separated and the aqueous layer was back extracted with methylene chloride (45 L). The combined product rich methylene chloride layers were then washed with water (91 L). The methylene chloride layer was then charged back into a clean reactor 2 and then concentrated to a volume of 64 k. Slowly charged ethyl acetate (185 L) to reactor 2 and the contents in reactor 2 were concentrated to 64 L. Repeated the ethyl acetate charge and concentrated one more time before cooling the reduced ethyl acetate product slurry in reactor 2 to 15° C. to 25° C. Granulated the contents in reactor 2 for 2.5 hours and then filtered. The filter cake was washed with ethyl acetate (34 L) and the product dried at 40° C. The melting point was 168° C. to 169° C.

We claim:

1. A process for preparing a compound of formula I 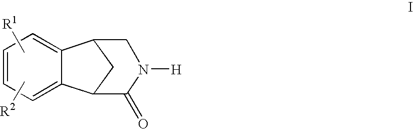
comprising hydrogenating a compound of formula II 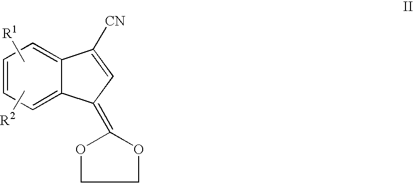
with hydrogen gas and an alcohol having the formula R³CH in the presence of a hydrogenation catalyst and an acid; wherein
R¹ and R² are selected independently from hydrogen, C₁-C₅ alkyl, C₁-C₅ alkoxy, trifluoromethyl, halogen, sulfonyl alkyl, alkyamino, amide, ester, aryl-alkyl, heteroalkyl and aryl-alkoxy;
of R¹ and R² together with the carbon atoms to which they are attached form a monocyclic or bicyclic ring; and R³ is C₁-C₅ alkyl; and
a. the hydrogenation catalyst is comprised of about 5% to about 10% of palladium on carbon;
b. the hydrogenation catalyst is present at a weight ratio of catalyst to compound of formula II in the range from about 1:99 to about 10:90;
c. the hydrogenation catalyst is comprised of about 30% to about 60% by weight of water;
d. the nitrile group is reduced to the corresponding amino group; and
e. the acid is present at an equivalence ratio of acid to the amino group of about 1:1.

2. The process according to claim 1 wherein
a. the hydrogenation of compounds of formula II leads to the formation of intermediate compounds of formula 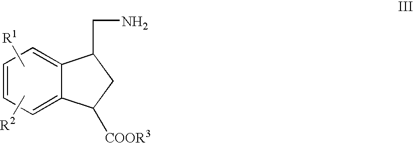
wherein R³ is C₁ to C₈ alkyl; and
b. the intermediate compounds of formula III are converted into compounds of formula I by treatment with a base in a solvent comprising an alcohol of formula R³OH, wherein R³ is C₁-C₆ alkyl.

3. The process according to claim 2 wherein the intermediate compounds of formula III are converted into compounds of formula I without prior isolation.

4. The process according to claim 2 wherein the intermediate compounds of formula III are isolated prior to conversion into compounds of formula I wherein R³ is C₃ to C₈ and the amino group is bound as an acid salt.

5. The process according to claim 1 wherein said hydrogenation catalyst is comprised of about 5% palladium on carbon.

6. The process according to claim 1 wherein said weight ratio of catalyst to compound of formula II is about 10:90.

7. The process according to claim 1 wherein said hydrogenation catalyst is comprised of about 50% by weight of water.

8. The process according to claim 1 wherein said acid is sulfuric add.

9. The process according to claim 1 wherein R³ is C₁ or C₂ alkyl.

10. The process according to claim 2 wherein said base is a Group I metal alkoxide.

11. The process according to claim 10 wherein said base is sodium tert-butoxide.

12. The process according to claim 2 wherein the alcohol contains a minimal quantity of water.

13. The process according to claim 1 wherein the compound of formula I is selected from the group consisting of
10-aza-tricyclo[6,3,1,0,2,7]dodeca-2,4,6-triene-9-one;
3trifluoromethyl-10-aza-tricyclo[6,3,1,0^{2,7}]dodeca-2(7),3,5-triene-9one;
(+)-3-trifluoromethyl-10-aza-tricyclo[6,3,1,0^{2,7}]dodeca-2(7),3,5-triene-9one;
(−)-3-trifluoromethyl-10-aza-tricyclo[6,3,1,0^{2,7}]dodeca-2(7),3,5-triene-9one;
3-fluoro-10-aza-tricyclo[6,3,1,0^{2,7}]dodeca-2(7),3,5-triene-9one;
(+)-3-fluoro-10-aza-tricyclo[6,3,1,0^{2,7}]dodeca-2(7),3,5-triene-9one; and
(−)-3-fluoro-10-aza-tricyclo[6,3,1,0^{2,7}]dodeca-2(7),3,5-triene.
